(19) United States
(12) Patent Application Publication
(10) Pub. No.: US 2016/0030222 A1
(43) Pub. Date: Feb. 4, 2016

(54) Ankle-Foot Flexion Device

(71) Applicant: ActivAided Orthotics LLC, Pittsburgh, PA (US)

(72) Inventors: Kelly N. Collier, Pittsburgh, PA (US); Gary P. Chimes, Redmond, WA (US); Jennifer A. Lambiase, Goshen, NY (US)

(73) Assignee: ACTIVAIDED ORTHOTICS LLC, Pittsburgh, PA (US)

(21) Appl. No.: 14/818,023

(22) Filed: Aug. 4, 2015

Related U.S. Application Data
(60) Provisional application No. 62/032,816, filed on Aug. 4, 2014.

(51) Int. Cl. A61F 5/01

(52) U.S. Cl. CPC A61F 5/0111; A61F 2005/0197

(57) ABSTRACT

The invention relates to an ankle-foot flexion device that is configurable to provide both plantar flexion and dorsiflexion support. A leading loop and base loop are adapted to engage the foot and ankle and can be constructed of elastic material. Tension straps provide a pulling force between the base loop and a leg cuff. The tension straps are adjustable and can be positioned according to the needs of a particular user.

### CROSS-REFERENCE TO RELATED APPLICATION

This application claims the benefit under 35 U.S.C. § 119 of U.S. Provisional Application Serial No. 62/032,816, filed Aug. 4, 2014, which is incorporated herein by reference.

### FIELD OF THE INVENTION

The present invention relates generally to a multi-purpose support garment. More specifically, the invention relates to an ankle-foot flexion device capable of being configured in various settings to assist with dorsiflexion or plantarflexion of the foot and ankle to achieve dynamic stretching and strengthening for the purpose of rehabilitation and conditioning.

### BACKGROUND

Lower extremity injuries, such as Achilles tendinopathy, affect active individuals and the aging population. These injuries can be characterized by inflammation and small tears of the tendons. Effective rehabilitative practices for treatment of Achilles tendon injuries, for example, include isometric and eccentric loading as well as stretching of the tendon-muscle complex. These practices apply tensile forces to the fibers in the tendon causing realignment of the fibers parallel to one another for a stronger structural unit.

Flexion is an important variable to control when treating the Achilles tendon. Dorsiflexion of the foot occurs as the foot decreases the angle made between it and the tibia, pointing the toe upwards towards the knee, which causes the Achilles tendon to elongate and stretch. Plantarflexion occurs when the foot is flexed away from the tibia, pointing the toe away from the body, which causes the Achilles tendon to shorten.

Currently, individuals suffering from tendon injuries in the ankle and foot are prescribed physical therapy sessions and/or a brace. Physical therapists instruct their patients in proper eccentric loading and dorsiflexion stretching practices. Braces are prescribed to aid the healing process and realign the damaged tissue within the tendon. For tendons around the ankle, the majority of these braces fall under the category of night splints. Night splints provide a static tensile force to the tendon-muscle complex, keeping it elongated during the user's sleep, a time in which one is otherwise inclined to keep a plantarflexed ankle position and shorten tendons such as the Achilles.

Eccentric loading exercises for rehabilitation include weighted motions where the foot is dorsiflexed and the calf muscles and Achilles tendon are lengthened. Patients are typically instructed to do these exercises dozens of times every day. Another common tool used as patients return to regular activity is a heel lift or wedge that increases the angle of plantarflexion between the foot and tibia when standing or walking. This prevents the Achilles tendon from being fully lengthened under a body weight load, decreasing the amount of force exerted on the tendon while standing or walking. While the physical therapy and braces are useful for rehabilitation, they do not allow constant ankle-foot support as they are only done intermittently.

It would therefore be advantageous to develop an ankle-foot flexion device that provides both plantarflexion and dorsiflexion support, where the device can be used during most daily activities.

### BRIEF SUMMARY OF THE INVENTION

According to embodiments of the present disclosure is a dynamic ankle-foot flexion device that uses variable resistance to stretch and strengthen the Achilles tendon and related muscles, tendons, and ligaments in the lower leg. The device is adapted to be used in either a dorsiflexion or plantarflexion configuration, depending on which is desired based on the demands of the activity undertaken or the preferences of the user. The configuration of the device is easily switched by changing the positioning a base loop, in one embodiment. In the dorsiflexion setting, adjustable elastic resistance straps attach at the foot to a leading loop through the base loop and at the lower leg, applying a force from underneath the foot to pull it towards the tibia. This assisted dorsiflexion results in light, long duration therapeutic stretching of the Achilles tendon and posterior muscles in the lower leg. In the plantarflexion setting, adjustable elastic resistance straps attach at the foot through the base loop and at the lower leg, applying a force from the top of the foot to point it away from the tibia. This assisted plantarflexion results in a decreased force required for propulsion while walking or running, as well as causing eccentric loading when the Achilles lengthens during the swing and foot strike phases of gait. Variable resistances are used to apply the appropriate amount of force in the necessary flexion direction, and strap placement is adjustable for optimal positioning and tension for each user.

The dynamic nature of the elastic resistance straps, compared to non-stretch straps, is significant. In the dorsiflexion setting, the elasticity of the straps makes the device a functional brace, which allows for walking and natural activity, while also offering the same stretching benefit of night splints when at rest or during periods of inactivity. In the plantarflexion setting, the elasticity of the straps enables the device to assist with the concentric contraction of the calf muscles when the foot is plantarflexed, while also acting as an increasing force on the foot when the tendon/muscle bodies are lengthened as the foot is dorsiflexed. Applying a force to a muscle as it is lengthened is an example of eccentric loading, which is a common treatment method for Achilles tendon injuries.

The present invention allows users to rehabilitate or condition as they go about normal daily activities, whereas many current braces on the market only focus on rehabilitation during periods of rest. In addition to the use of elastic resistance straps that enable walking and natural activity in some embodiments, the elastic resistance straps are low profile and lay close to the foot and lower leg, which is conducive to use with regular shoes and attire. As a result, the device avoids the obstacles with traditional braces and taping, such as the inability to use with normal shoes, motion restriction caused by non-stretch strapping/tape, the need to frequently reapply tape, and the inability of non-stretch strapping/tape to allow for dynamic, eccentric loading. Additionally, the multiple settings allow users to maintain an active lifestyle by offering them the option to switch the device into the plantarflexion mode, which decreases the load experienced in the calf muscles and Achilles tendon while walking or running, allowing the patient to return to normal activity sooner without causing further injury.

The advantages of using the ankle-foot flexion device in the plantarflexion setting include off-loading the Achilles tendon and decreasing the forces experienced in the Achilles tendon and posterior muscles of the lower leg while walking, running, or jumping. This occurs due to the tension in the elastic strap extensions which aids flexion in the plantarflexion direction, and adds resistance in the dorsiflexion direction. By aiding plantarflexion motion in the propulsion phase of walking or running, patients rehabilitating from Achilles tendinopathy or other related disorders can return to typical activity more quickly. Additionally, by adding resistance to the dorsiflexion motion while walking or running, the Achilles tendon and posterior muscles undergo eccentric loading where force is added while the muscles lengthen. Eccentric loading has been proven to be an effective therapeutic technique for Achilles tendinopathy.

The advantages of using the ankle-foot flexion device in the dorsiflexion setting include passive, low load stretching of the Achilles tendon and calf muscles over a moderately long duration. High load stretching, such as with the entire body weight, can cause tears and additional damage to the Achilles tendon due to the excessive force experienced in the tendon. While a patient is sitting, lying, or otherwise off their feet, this light stretching over a period of greater than 5 minutes up to 8 hours can allow the tendon fibers to elongate and realign in their preferred orientation as seen in a healthy Achilles tendon.

In addition to the novelty of a dual mode device including both a plantarflexion setting and dorsiflexion setting, the present invention ankle-foot flexion device has the unique ability to be worn inside of a typical shoe and does not restrict from common daily habits or activities including walking, sitting, standing, or athletic activities.

### BRIEF DESCRIPTION OF THE DRAWINGS

FIG. 1 is a front view of the ankle-foot flexion device in the plantarflexion setting according to one embodiment.

FIG. 2 is a left side view of the ankle-foot flexion device in the plantarflexion setting.

FIG. 3 is a back view of the ankle-foot flexion device in the plantarflexion setting.

FIG. 4 is a right side view of the ankle-foot flexion device in the plantarflexion setting.

FIG. 5 is a top view of the ankle-foot flexion device in the plantarflexion setting.

FIG. 6 is a bottom view of the ankle-foot flexion device in the plantarflexion setting.

FIG. 7 is a front view of the ankle-foot flexion device in the plantarflexion setting showing the adjustability of certain components of the device.

FIG. 8 is a front view of the ankle-foot flexion device in the dorsiflexion setting.

FIG. 9 is a left side view of the ankle-foot flexion device in the dorsiflexion setting.

FIG. 10 is a back view of the ankle-foot flexion device in the dorsiflexion setting.

FIG. 11 is a right side view of the ankle-foot flexion device in the dorsiflexion setting.

FIG. 12 is a top view of the ankle-foot flexion device in the dorsiflexion setting.

FIG. 13 is a bottom view of the ankle-foot flexion device in the dorsiflexion setting.

FIG. 14 is a front view of the ankle-foot flexion device in the dorsiflexion setting showing the adjustability of certain components of the device.

FIG. 15A shows the leg cuff uncoupled from the strap extensions.

FIG. 15B is a front view of the ankle-foot flexion device in the plantarflexion setting, showing both detachable strap extensions disengaged from their releasably attached state.

FIG. 16A shows the leg cuff according to an alternative embodiment, with friction material disposed on the inner surface of the cuff.

FIG. 16B is a front view of the ankle-foot flexion device in the dorsiflexion setting showing both detachable strap extensions disengaged from their releasably attached state.

FIG. 17 is a right side view of the ankle-foot flexion device in the dorsiflexion setting according to an alternative embodiment where the strap extensions may attach on one end to the front of the foot and on the other end to a calf sleeve, where the straps are held in place underneath a guiding ankle cuff.

FIG. 18 is a right side view of the ankle-foot flexion device in the dorsiflexion setting according to the embodiment depicted in FIG. 17 where the strap extensions may attach directly to a traditional shoe.

### DETAILED DESCRIPTION OF THE INVENTION

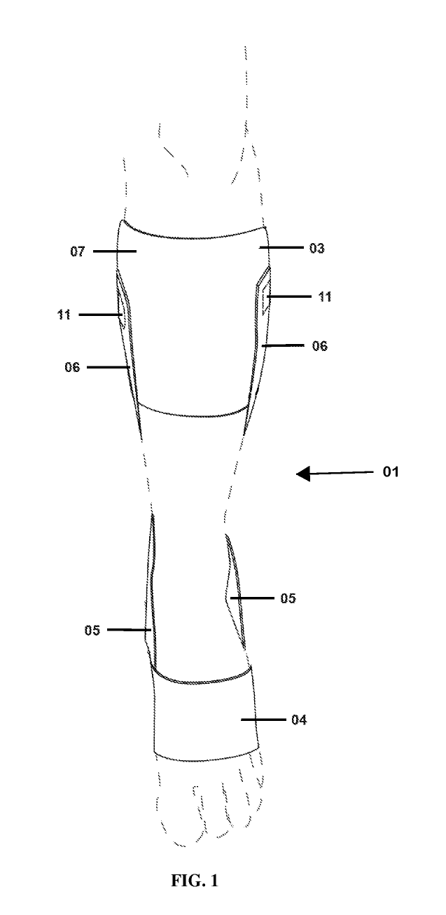

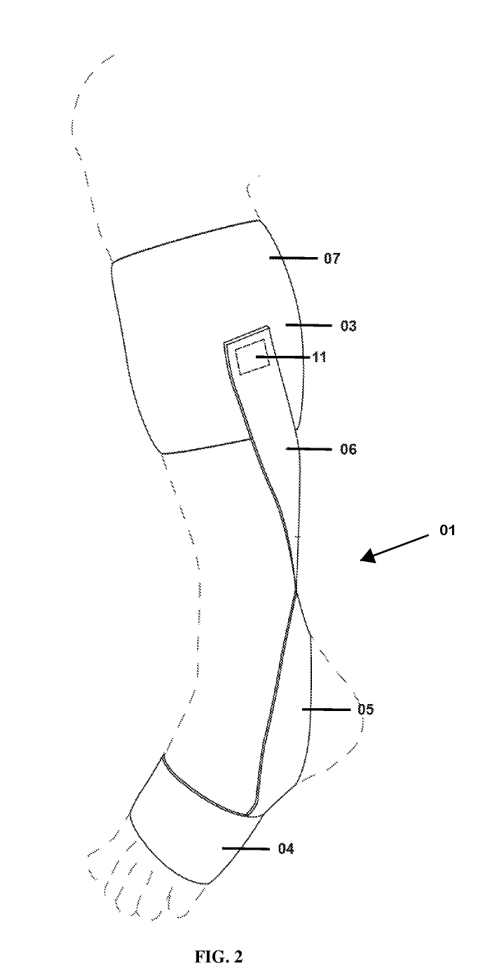

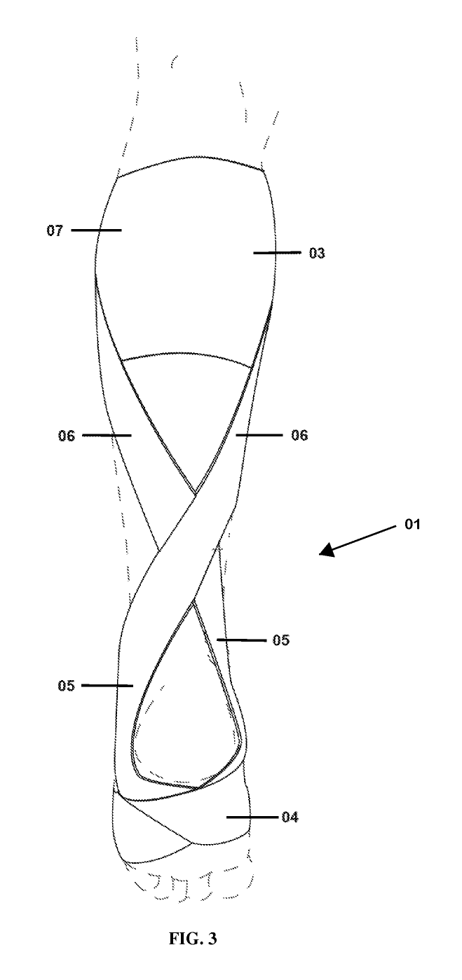

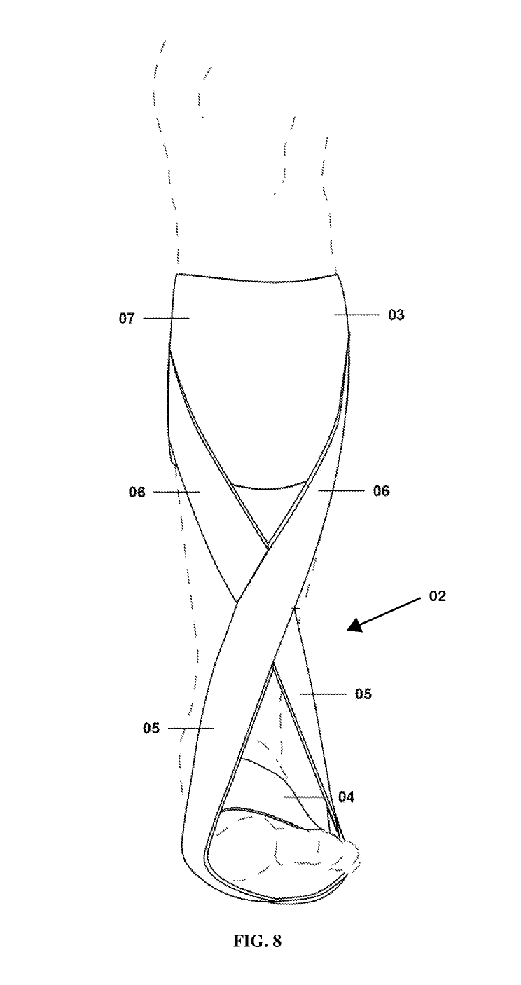

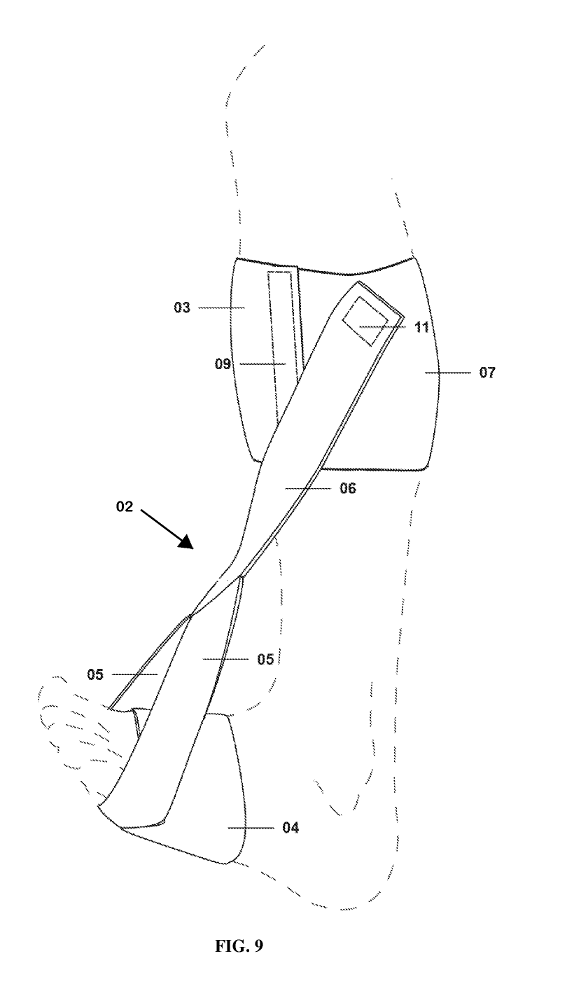

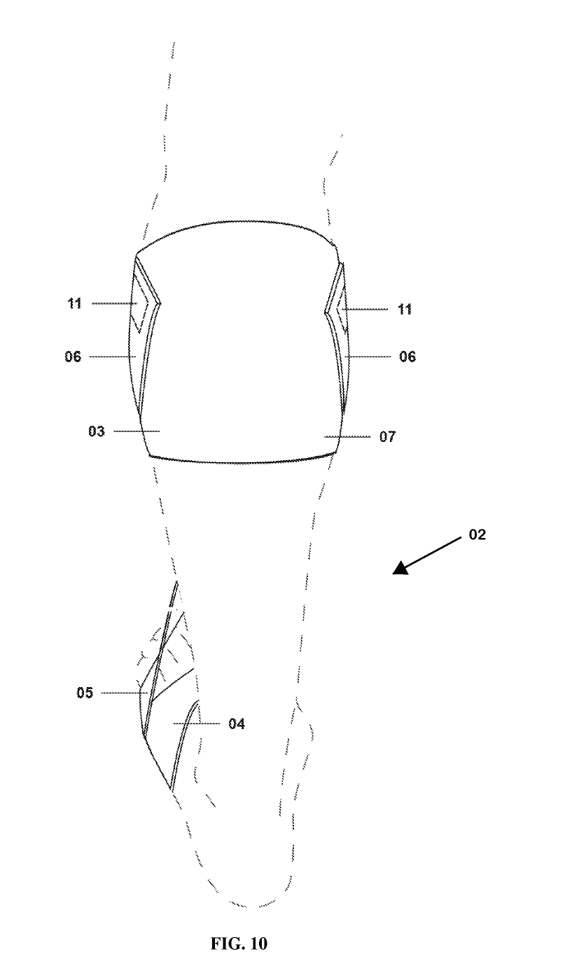

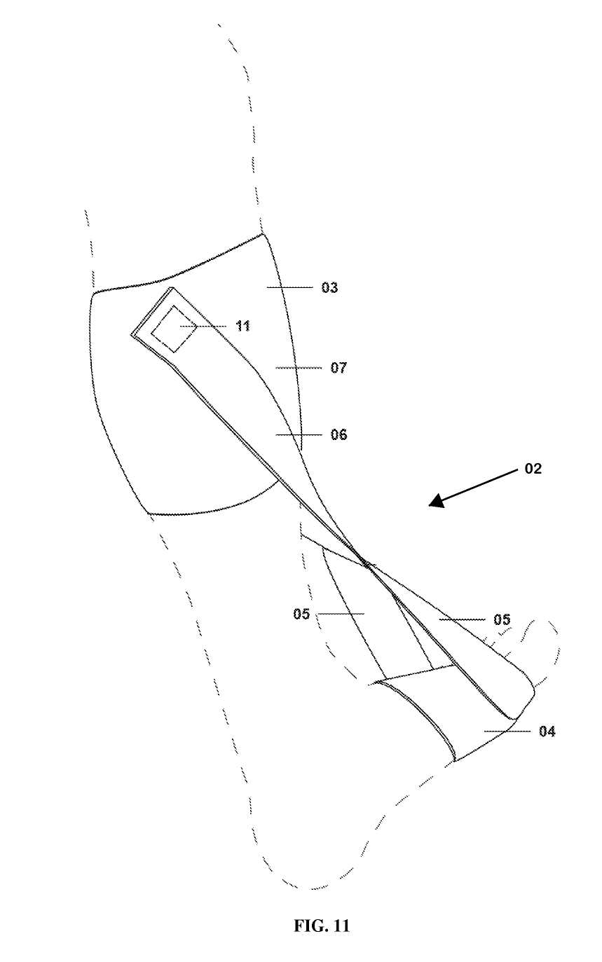

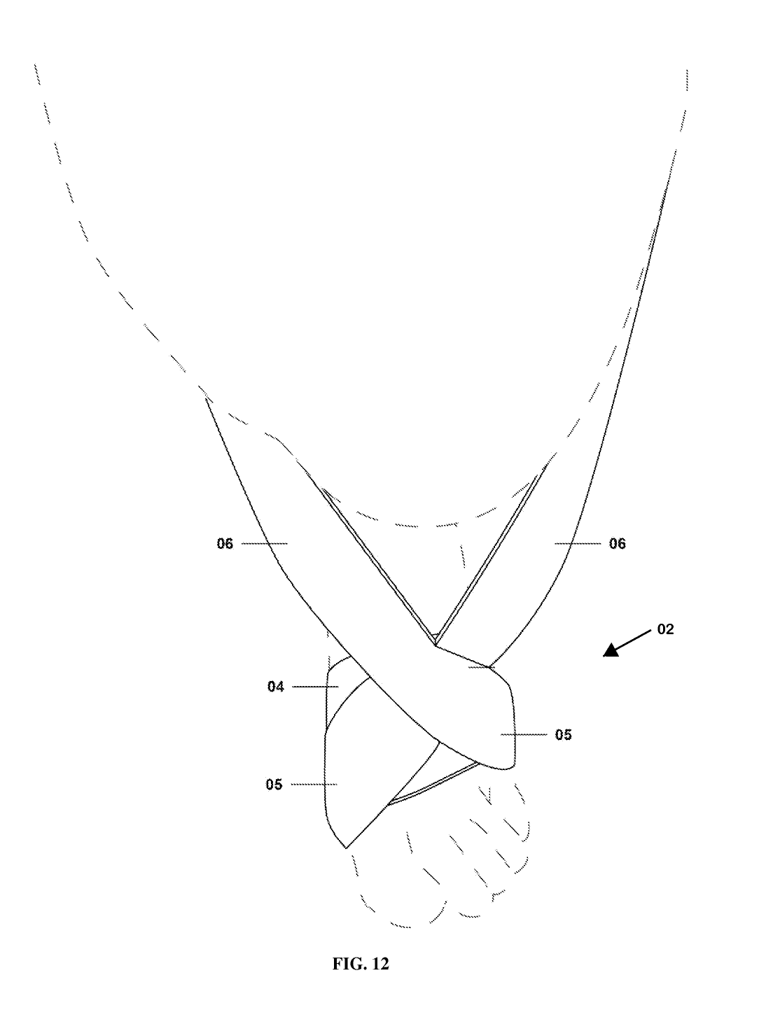

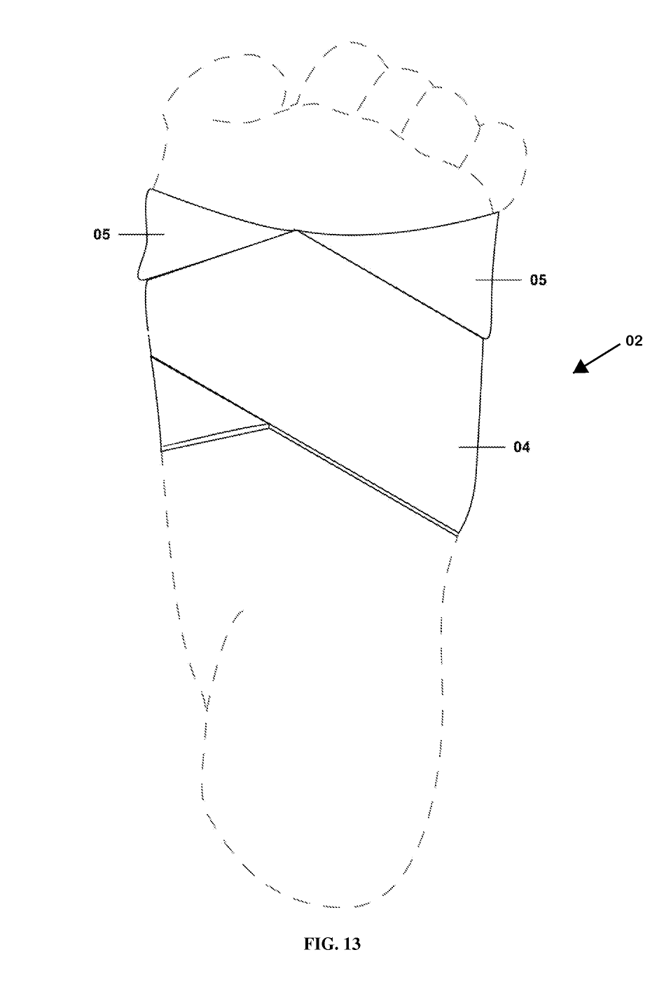

Embodiments of the present invention and its advantages are best understood by referring to the figures. FIGS. 1-6 are alternate views of the device according to a preferred embodiment and illustrate a plantarflexion configuration 01. FIGS. 8-13 are alternate views that show the device in a dorsiflexion configuration 02. As can be seen in FIG. 1, leading loop 04 is adapted to engage an anterior portion of the foot. The leading loop 04 can be constructed of an inelastic material sized to a user's foot or it can be made of an elastic material to encompass a broader range of sizes. A base loop 05 extends from the leading loop 04 and its orientation depends on the particular configuration. In the plantarflexion configuration 01, the base loop 05 extends from the leading loop 04 towards the back of the foot and around the heel. As shown in FIGS. 2-3, the base loop 05 terminates near the Achilles tendon when providing plantarflexion support.

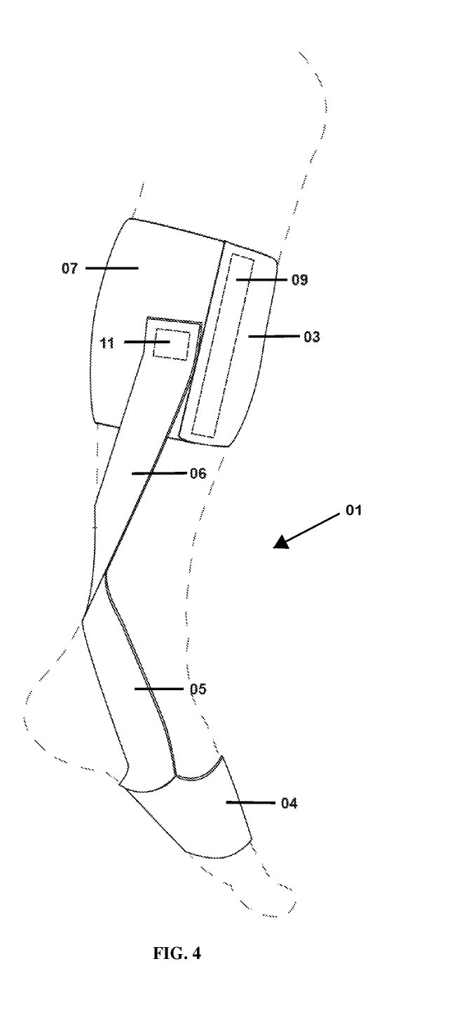

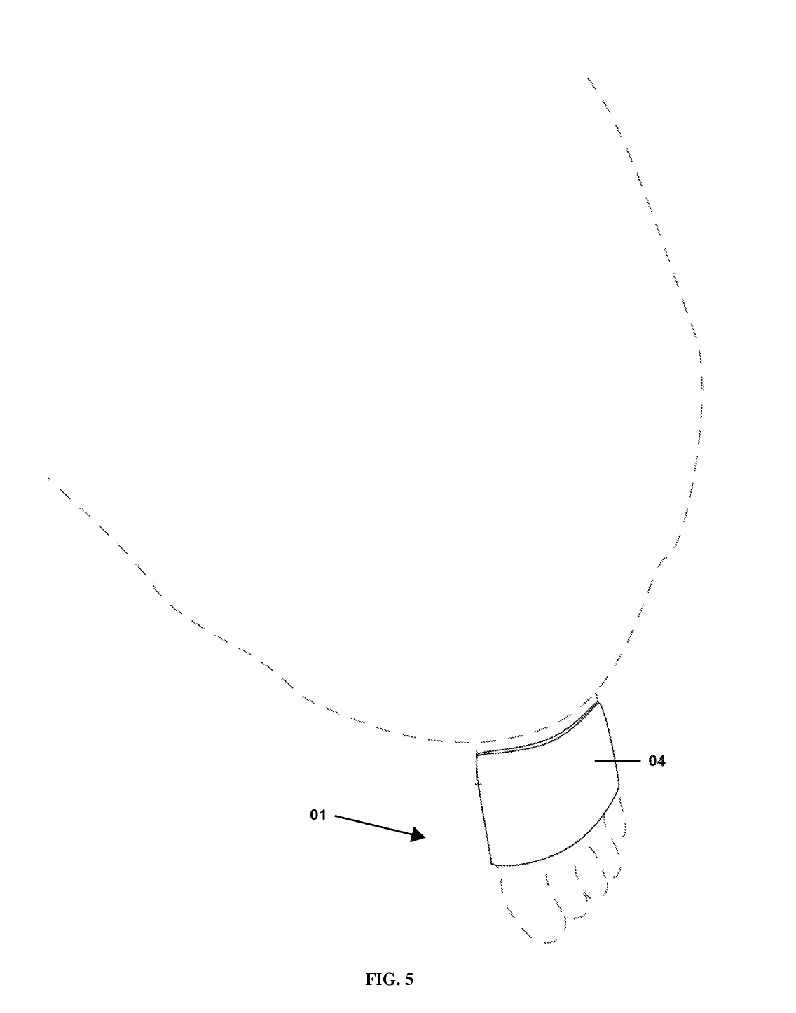

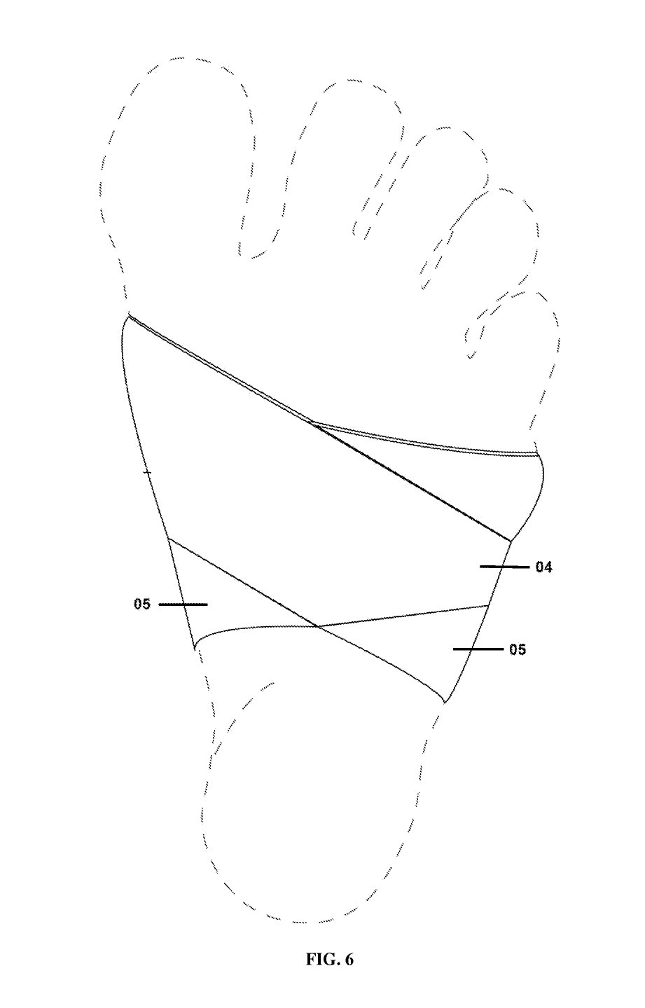

A plurality of strap extensions 06 extend from the base loop 05. The extensions, or tension straps 06, may releasably attach to a leg cuff 03 with extension hook 11. In the plantarflexion configuration 01, the tension straps 06 extend from the base loop 05 along the back of the leg to the leg cuff 03, as shown in FIG. 3. Referring to FIG. 4, it can be seen that the device in the plantarflexion configuration 01 tends to put a downward force on the foot, extending the angle between the top of the foot and the tibia. FIGS. 5-6 show the low-profile nature of the device, allowing a user to wear the device beneath a pair of shoes for all-day use.

In the preferred embodiment, the leading loop 04, base loop 05, and strap extension 06 are made from an elastomeric material where each component may have different elasticities. For example, the leading loop 04 may be constructed of a less elastic material than the strap extensions 06 to aid in comfort around the foot, while allowing the strap extensions 06 to provide sufficient tension. In a preferred embodiment, the leg cuff 03 is made from an elastomeric material with an outer surface of loop material 07 which is receptive to a cooperating hook material 09 present on the inner surface of the leg cuff 03 and inner surface of the strap extension 06, shown as extension hook 11 in some embodiments, so that they can be releasably attached to the outer surface loop 07 of the leg cuff 03. However, other closure and attachment mechanisms can be used in alternative embodiments. To keep the device from slipping when worn, an inner surface gripping material 08 and 10, such as silicone, can be disposed on the leg cuff 03 and leading loop 04, respectively, to create friction.

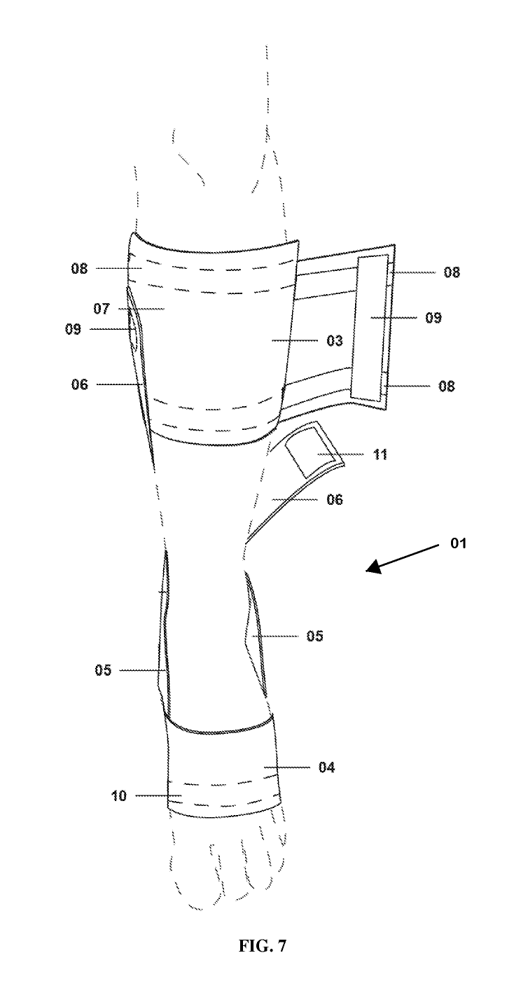

To use the device, a user places the leading loop 04 over the front, or anterior portion, of the foot. The leg cuff 03 is preferentially positioned below the knee and above the calf. As shown in FIG. 7, the leg cuff 03 is wrapped around the leg and closed by fastening the hook material 09 to the outer surface loop material 09. In the preferred embodiment, where the leg cuff is made from elastomeric material, a slight compressive force is imparted on the leg by the cuff. Because the cuff is adjustable, the user can tighten or loosen the cuff as necessary for comfort or to prevent the cuff from sliding down the leg. In an alternative embodiment, the leg cuff 03 can be secured on a different portion of the leg, such as the lower leg.

After securing the leading loop 04 and the leg cuff 03, the base loop 05 is positioned to extend around the heel if used in the plantarflexion configuration 01. The user then pulls the strap extensions 06 upwards, behind the ankle, and attaches them to the leg cuff 03. Hook material disposed on the end of the strap extension 06 allows the user to attach the end to any part of the leg cuff 03, which has an exterior surface made of loop material. The strap extensions 06 are pulled into tension, causing a force to be applied to the top of the anterior portion of the foot by way of the leading loop 04 and pulls the foot in the direction of plantarflexion. The tension in the strap extensions 06 can be adjusted based on personal preference or until the angle of plantarflexion while using the flexion device is greater than the angle of plantarflexion of the foot at rest when not using the ankle-foot flexion device. In the preferred embodiment, the entire surface of the leg cuff 03 is comprised of loop material 07 to allow for variability in tension and position of attachment of the strap extensions 06. Referring again to FIG. 7, illustrated is how the hook material 09 on the leg cuff 03 and the hook material 09 on one of the strap extensions 06 is disengaged from the outer surface loop 07 using a hook and loop engagement system.

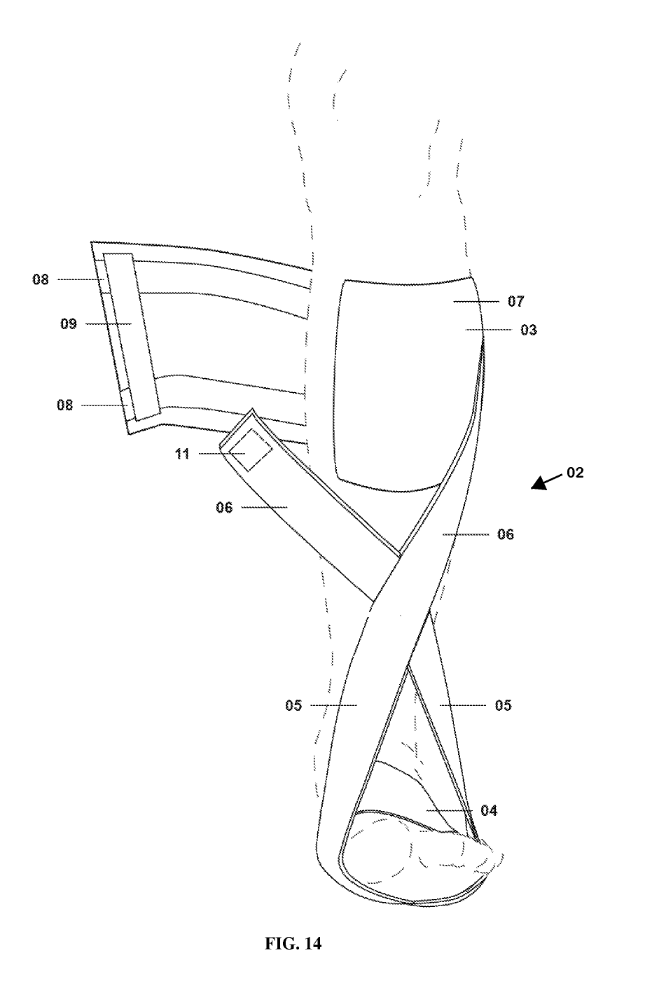

FIGS. 8-14 are alternate views illustrating the device of the present invention in a dorsiflexion setting 02. In this setting, the leg cuff 03 and leading loop 04 are positioned in similar positions as they are in the plantarflexion configuration 01. However, in the dorsiflexion 02 configuration, the base loop 05 encircles the anterior portion of the foot and the strap extensions 06 extend upwards in front of the ankle towards the leg cuff 03. The strap extensions 06 are pulled into tension, causing a force to be applied to the bottom of the anterior portion of the foot by way of the leading loop 04 and pulls the foot in the direction of dorsiflexion. As with the plantarflexion configuration 01, the tension in the strap extensions 06 can be adjusted based on personal preference or until the angle of dorsiflexion while using the flexion device is greater than the angle of dorsiflexion at rest when not using the ankle-foot flexion device. FIG. 14 illustrates how the hook material 09 on the leg cuff 03 and the hook material 09 on one of the strap extensions 06 is disengaged from the outer surface loop 07 using a hook and loop engagement system.

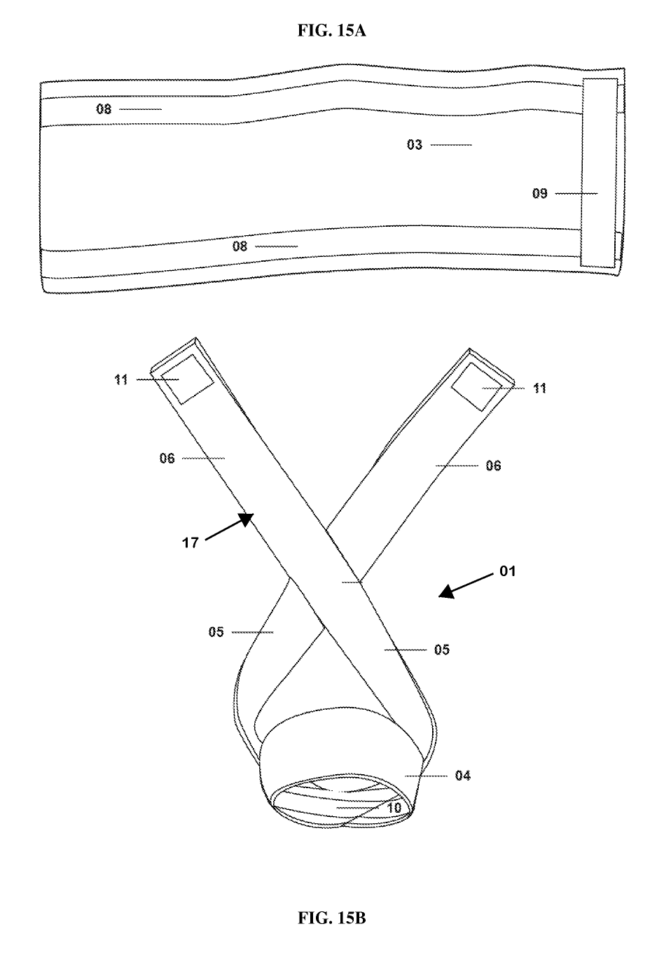

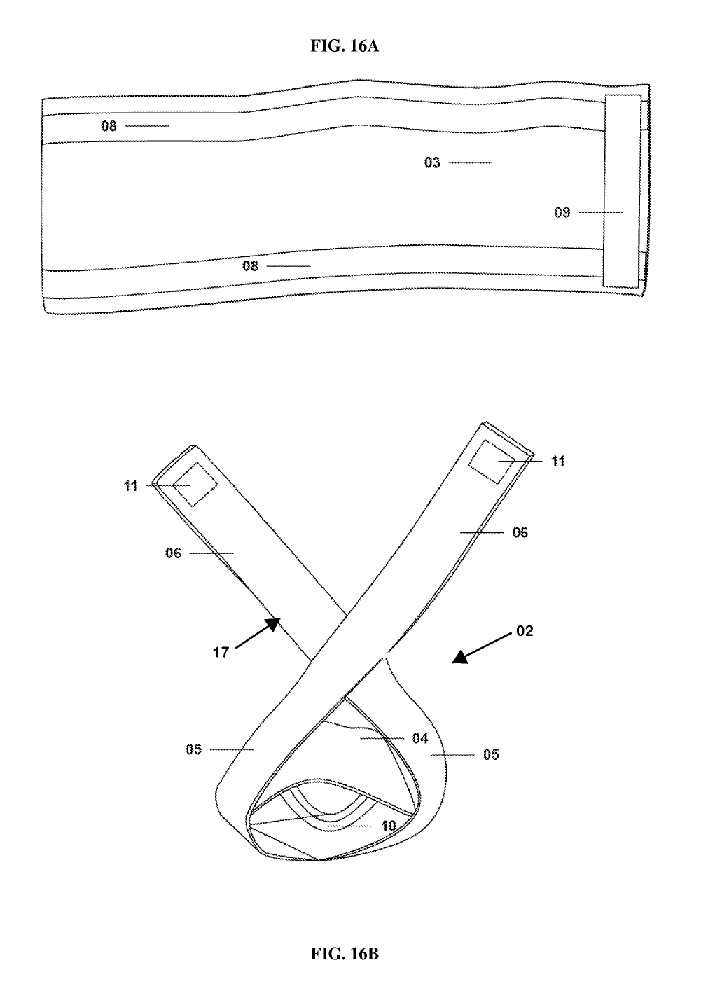

FIG. 15A-B and 16A-B illustrate one particular embodiment in which the leading loop 04, base loop 05, and extension straps 06 are constructed from a single piece of strapping 17. In this embodiment, the leading loop 04 is formed near the middle of the strapping 17 and joined together, either freely or permanently, such as by sewing. The strapping 17 extends from the leading loop 04 and is crossed at a junction, forming the base loop 05. The strapping 17 can be joined freely or permanently at the junction. The strapping 17 extends from the junction, forming the extension straps 06, with the ends of the extension straps 06 coinciding with the ends of the strapping 17. The inner surface of the strap extensions 06 near the farthest end from the junction includes hook material 09 on each of the two strap extensions 06. The leg cuff 03 is constructed from a length of elastomeric strap including hook material 09 on one end of the strap.

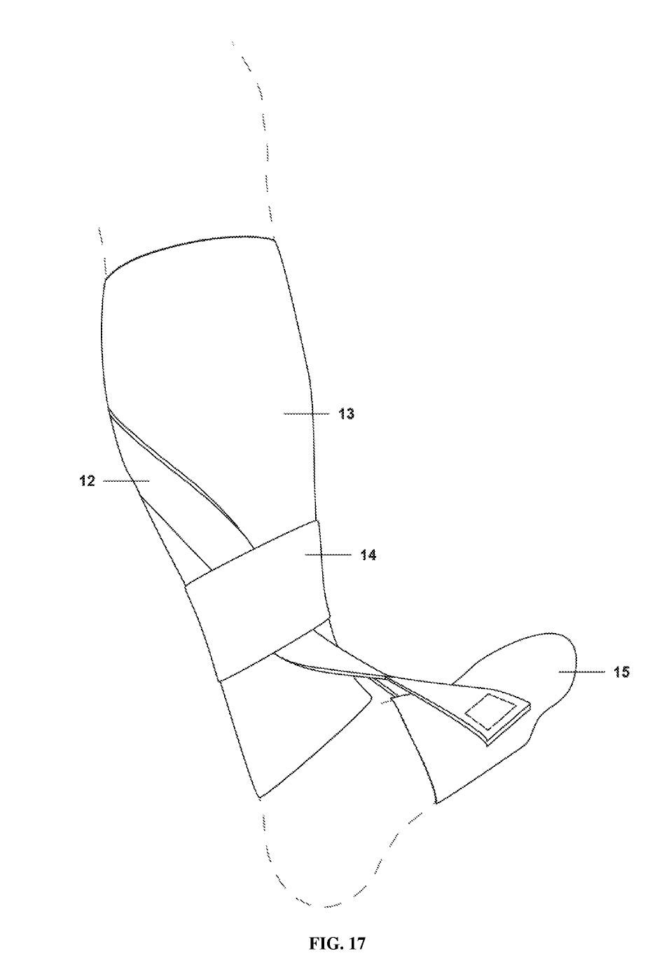

FIG. 17 illustrates an alternative embodiment of the present invention in the dorsiflexion setting where the strap extensions 12 with no leading loop or base loop may releasably attach on one end to a toe cap 15 encompassing the front of the foot and releasably attach on the other end to a calf sleeve 13. The strap ends 12 are held in place by being routed underneath a guiding ankle cuff 14.

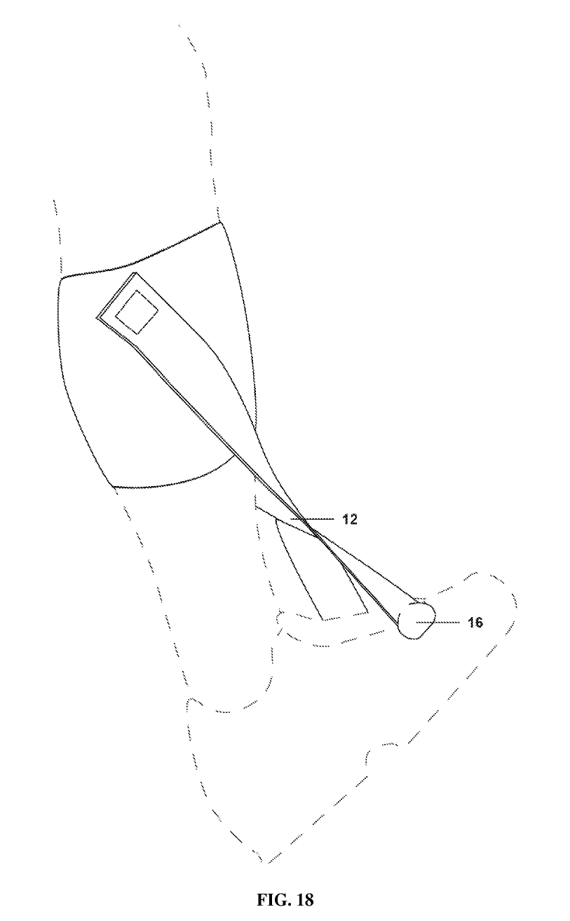

FIG. 18 illustrates an alternative embodiment of the present invention in the dorsiflexion setting where the strap extensions 12 with no leading loop or base loop may releasably attach on one end to the leg cuff 03 and releasably attach on the other end directly to the outside of a shoe through the use of shoe clips 16.

While the disclosure has been described in detail and with reference to specific embodiments thereof, it will be apparent to once skilled in the art that various changes and modifications can be made therein without departing from the spirit and scope of the embodiments. Thus, it is intended that the present disclosure cover the modifications and variations of this disclosure provided they come within the scope of the appended claims and their equivalents.

What is claimed is:

1. An ankle-foot flexion device for providing plantarflexion or dorsiflexion support, the flexion device comprising:
a leading loop adapted to engage an anterior portion of the foot;
a base loop extending from the leading loop and having a first configuration wherein the base loop is adapted to provide plantar flexion support and a second configuration wherein the base loop is adapted to provide dorsiflexion support;
a leg cuff adapted to engage a portion of the leg;
a plurality of tension straps connecting the base loop to the leg cuff,
wherein each of the plurality of straps has an end,
wherein the end is positionable on the leg cuff, thereby allowing adjustment of a tension force created by the plurality of straps between the leg cuff and the base loop.

2. The ankle-foot flexion device of claim 1, wherein the base loop comprises:
a strap having a first end and a second end,
base loop strap at the junction.

4. The ankle-foot flexion device of claim 1:
wherein the base loop is constructed of a first elastic material having a first elasticity;
wherein the leading loop is constructed of a second elastic material having a second elasticity;
wherein the first elasticity and the second elasticity are not equal.

5. The ankle-foot flexion device of claim 1, further comprising:
an anti-slip material disposed on an inner surface of the leading loop and an inner surface of the leg cuff.

6. The ankle-foot flexion device of claim 1, wherein the plurality of straps are constructed of an elastic material.

7. The ankle-foot flexion device of claim 1, wherein the tension force does not inhibit ambulation.

8. The ankle-foot flexion device of claim 1, wherein the base loop and leading loop are adapted to be worn inside a shoe.

9. The ankle-foot flexion device of claim 1, wherein the based loop positioned in the first configuration is adapted to engage a posterior portion of the foot and to extend around the heel, terminating near the Achilles tendon.

10. The ankle-foot flexion device of claim 1, wherein the based loop positioned in the second configuration does not directly engage the foot or the ankle.

11. The ankle-foot flexion device of claim 1, wherein the leading loop substantially cover the anterior portion of the foot.

12. An ankle-foot flexion device for providing plantarflexion or dorsiflexion support, the flexion device comprising:
a leading loop adapted to engage an anterior portion of the foot;
a leg cuff adapted to engage a portion of the leg; and
a plurality of tension straps connecting the leading loop to the leg cuff,
wherein the plurality of tension straps are positionable in a first configuration providing plantarflexion support and a second configuration providing dorsiflexion support,
wherein each of the plurality of straps has an end,
wherein the end is positionable on the leg cuff, thereby allowing adjustment of a tension force created by the plurality of straps between the leg cuff and the base loop.

13. An ankle-foot flexion device for providing plantarflexion or dorsiflexion support, the flexion device comprising:
a leg cuff adapted to engage a portion of the leg; and
a support strap connected to the leg cuff, the support strap comprising:
a leading loop adapted to engage an anterior portion of the foot, wherein the leading loop is formed by looping the strap and affixing the loop to itself at a junction;
a base loop extending from the leading loop and having a first configuration providing plantar flexion support and a second configuration providing dorsiflexion support,
wherein the base loop is formed by crossing a first portion and a second portion of the support strap extending from the leading loop;
an pair of extensions connecting the base loop to the leg cuff,
wherein each of the pair of extensions has an end coinciding with each of a pair of ends of the support strap,
wherein each of the end of the pair of extensions is positionable on the leg cuff, thereby allowing adjustment of a tension force created by the pair of extensions between the leg cuff and the base loop.

14. The ankle-foot flexion device of claim 13, wherein the leg cuff is a substantially covers a portion of the leg below the knee.

15. The ankle-foot flexion device of claim 14, further comprising:
a guiding cuff adapted to engage a portion of the leg,
wherein the guiding cuff is positioned over the leg cuff and the pair of extensions, thereby guiding the pair of extensions against the portion of the leg.
